(19) United States
(12) Patent Application Publication
(10) Pub. No.: US 2008/0280842 A1
(43) Pub. Date: Nov. 13, 2008

(54) Fluorinated Pyrrolo[2,3-D]Pyrimidine Nucleosides for the Treatment of Rna-Dependent Rna Viral Infection

(75) Inventors: Malcolm MacCoss, Freehold, NJ (US); David B. Olsen, Lansdale, PA (US); Joseph Leone, Tinton Falls, NJ (US); Philippe L. Durette, New Providence, NJ (US)

Correspondence Address: MERCK AND CO., INC, P O BOX 2000, RAHWAY, NJ, 07065-0907, US

(73) Assignee: MERCK & CO., INC., Rahway, NJ (US)

(21) Appl. No.: 11/664,564

(22) PCT Filed: Oct. 17, 2005
(86) PCT No.: PCT/US05/37224

§ 371 Date: Apr. 3, 2007

Related U.S. Application Data
(60) Provisional application No. 60/620,743, filed on Oct. 21, 2004.
(60) Provisional application No. 60/651,366, filed on Feb. 9, 2005.

(51) Int. Cl. A61K 31/7064; C07H 19/04; A61P 31/12

(52) U.S. Cl. 514/43; 536/27.2

(57) ABSTRACT

The present invention provides fluorinated pyrrolo[2,3,d]pyrimidine nucleoside compounds which are inhibitors of RNA-dependent RNA viral polymerase. These compounds are inhibitors of RNA-dependent RNA viral replication and are useful for the treatment of RNA-dependent RNA viral infection. They are particularly useful as precursors to inhibitors of hepatitis C virus (HCV) NS5B polymerase, as precursors to inhibitors of HCV replication, and/or for the treatment of hepatitis C infection. The invention also describes pharmaceutical compositions containing such fluorinated pyrrolo[2,3-d]pyrimidine nucleoside alone or in combination with other agents active against RNA-dependent RNA viral infection, in particular HCV infection. Also disclosed are methods of inhibiting RNA-dependent RNA polymerase, inhibiting RNA-dependent RNA viral replication, and/or treating RNA-dependent RNA viral infection with the fluorinated pyrrolo[2,3-d]pyrimidine nucleoside of the present invention.

### FIELD OF THE INVENTION

The present invention is concerned with fluorinated pyrrolo[2,3-d]pyrimidine nucleoside compounds and certain derivatives thereof, their synthesis, and their use as inhibitors of RNA-dependent RNA viral polymerase. The compounds of the present invention are inhibitors of RNA-dependent RNA viral replication and are useful for the treatment of RNA-dependent RNA viral infection. They are particularly useful as inhibitors of hepatitis C virus (HCV) NS5B polymerase, as inhibitors of HCV replication, and for the treatment of hepatitis C viral infection.

### BACKGROUND OF THE INVENTION

Hepatitis C virus (HCV) infection is a major health problem that leads to chronic liver disease, such as cirrhosis and hepatocellular carcinoma, in a substantial number of infected individuals, estimated to be 2-15% of the world's population. There are an estimated 3.9 million infected people in the United States alone, according to the U.S. Center for Disease Control, roughly five times the number of people infected the human immunodeficiency virus (HIV). According to the World Health Organization, there are more than 170 million infected individuals worldwide, with at least 3 to 4 million people being infected each year. Once infected, about 20% of people clear the virus, but the rest harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their off-spring. Current treatments for HCV infection, which are restricted to immunotherapy with recombinant interferon-α alone or in combination with the nucleoside analog ribavirin, are of limited clinical benefit. Moreover, there is no established vaccine for HCV. Consequently, there is an urgent need for improved therapeutic agents that effectively combat chronic HCV infection. The state of the art in the treatment of HCV infection has been reviewed, and reference is made to the following publications: B. Dymock, et al., “Novel approaches to the treatment of hepatitis C virus infection,” Antiviral Chemistry & Chemotherapy, 11: 79-96 (2000); H. Rosen, et al., “Hepatitis C virus: current understanding and prospects for future therapies,” Molecular Medicine Today, 5: 393-399 (1999); D. Moradpour, et al., “Current and evolving therapies for hepatitis C,” European J. Gastroenterol. Hepatol., 11: 1189-1202 (1999); R. Bartenschlager, “Candidate Targets for Hepatitis C Virus-Specific Antiviral Therapy,” Intervirology, 40: 378-393 (1997); G. M. Lauer and B. D. Walker, “Hepatitis C Virus Infection,” N. Engl. J. Med., 345: 41-52 (2001); B. W. Dymock, “Emerging therapies for hepatitis C virus infection,” Emerging Drugs, 6: 13-42 (2001); and C. Crabb, “Hard-Won Advances Spark Excitement about Hepatitis C,” Science: 506-507 (2001); the contents of all of which are incorporated by reference herein in their entirety.

Different approaches to HCV therapy have been taken, which include the inhibition of viral serine proteinase (NS3 protease), helicase, and RNA-dependent RNA polymerase (NS5B), and the development of a vaccine.

The HCV virion is an enveloped positive-strand RNA virus with a single oligoribonucleotide genomic sequence of about 9600 bases which encodes a polyprotein of about 3,010 amino acids. The protein products of the HCV gene consist of the structural proteins C, E1, and E2, and the non-structural proteins NS2, NS3, NS4A and NS4B, and NS5A and NS5B. The nonstructural (NS) proteins are believed to provide the catalytic machinery for viral replication. The NS3 protease releases NS5B, the RNA-dependent RNA polymerase from the polyprotein chain. HCV NS5B polymerase is required for the synthesis of a double-stranded RNA from a single-stranded viral RNA that serves as a template in the replication cycle of HCV. NS5B polymerase is therefore considered to be an essential component in the HCV replication complex [see K. Ishi, et al., “Expression of Hepatitis C Virus NS5B Protein: Characterization of Its RNA Polymerase Activity and RNA Binding,” Hepatology, 29: 1227-1235 (1999) and V. Lohmann, et al., “Biochemical and Kinetic Analyses of NS5B RNA-Dependent RNA Polymerase of the Hepatitis C Virus,” Virology, 249: 108-118 (1998)]. Inhibition of HCV NS5B polymerase prevents formation of the double-stranded HCV RNA and therefore constitutes an attractive approach to the development of HCV-specific antiviral therapies.

The development of inhibitors of HCV NS5B polymerase with potential for the treatment of HCV infection has been reviewed in M. P. Walker et al., “Promising candidates for the treatment of chronic hepatitis C,” Expert Opin. Invest. Drugs, 12: 1269-1280 (2003); P. Hoffmann et al., “Recent patents on experimental therapy for hepatitis C virus infection (1999-2002),” Expert Opin. Ther. Patents,” 13: 1707-1723 (2003); and V. Brass, et al., “Recent developments in target identification against HCV,” Expert Opin. Ther. Targets,” 8: 295-307 (2004). Inhibition of HCV replication by purine ribonucleosides was reported by A. E. Eldrup, et al., in “Structure-Activity Relationship of Purine Ribonucleosides for Inhibition of HCV RNA-Dependent RNA Polymerase,” J. Med. Chem., 47: 2283-2295 (2004). There is a continuing need for structurally diverse nucleoside derivatives as inhibitors of HCV polymerase as therapeutic approaches for HCV therapy.

U.S. Pat. No. 6,777,396 (issued Aug. 17, 2004) disclosed a series of structurally novel pyrrolo[2,3-d]pyrimidine nucleoside derivatives as inhibitors of HCV NS5B polymerase useful for the treatment of HCV infection. The biological properties of such compounds were described by D. B. Olsen et al., in “A 7-Deaza-Adenosine Analog is a Potent and Selective Inhibitor of HCV Replication with Excellent Pharmacokinetic Properties,” Antimicrob. Agents Chemother., 48: 3944-3953 (2004) and by A. E. Eldrup, et al., in “Structure-Activity Relationship of Heterobase-Modified 2′-C-Methyl Ribonucleosides as Inhibitors of Hepatitis C Virus RNA Replication,” J. Med. Chem., 47: 5284-5297 (2004). It has now been found that introduction of fluorine at the C-5 position of the pyrrolo[2,3-d]pyrimidine nucleus (7-deaza-7-fluoroadenosine analogs) provides nucleoside derivatives which are more potent inhibitors of HCV RNA replication with superior pharmacokinetic properties, such as better distribution to the liver.

It is therefore an object of the present invention to provide fluorinated pyrrolo[2,3-d]pyrimidine nucleoside compounds and certain derivatives thereof which are useful as inhibitors of RNA-dependent RNA viral polymerase and in particular as inhibitors of HCV NS5B polymerase.

It is another object of the present invention to provide fluorinated pyrrolo[2,3-d]pyrimidine nucleoside compounds and certain derivatives thereof which are useful as inhibitors of the replication of an RNA-dependent RNA virus and in particular as inhibitors of the replication of hepatitis C virus.

It is another object of the present invention to provide fluorinated pyrrolo[2,3-d]pyrimidine nucleoside compounds and certain derivatives thereof which are useful in the treatment of RNA-dependent RNA viral infection and in particular in the treatment of HCV infection.

It is another object of the present invention to provide pharmaceutical compositions comprising the fluorinated pyrrolo[2,3-d]pyrimidine nucleoside compounds of the present invention in association with a pharmaceutically acceptable carrier.

It is another object of the present invention to provide pharmaceutical compositions comprising the fluorinated pyrrolo[2,3-d]pyrimidine nucleoside compounds and derivatives thereof of the present invention for use as inhibitors of RNA-dependent RNA viral polymerase and in particular as inhibitors of HCV NS5B polymerase.

It is another object of the present invention to provide pharmaceutical compositions comprising the fluorinated pyrrolo[2,3-d]pyrimidine nucleoside compounds and derivatives thereof of the present invention for use as inhibitors of RNA-dependent RNA viral replication and in particular as inhibitors of HCV replication.

It is another object of the present invention to provide pharmaceutical compositions comprising the fluorinated pyrrolo[2,3-d]pyrimidine nucleoside compounds and derivatives thereof of the present invention for use in the treatment of RNA-dependent RNA viral infection and in particular in the treatment of HCV infection.

It is another object of the present invention to provide pharmaceutical compositions comprising the fluorinated pyrrolo[2,3-d]pyrimidine nucleoside compounds and derivatives thereof of the present invention in combination with other agents active against an RNA-dependent RNA virus and in particular against HCV.

It is another object of the present invention to provide methods for the inhibition of RNA-dependent RNA viral polymerase and in particular for the inhibition of HCV NS5B polymerase.

It is another object of the present invention to provide methods for the inhibition of RNA-dependent RNA viral replication and in particular for the inhibition of HCV replication.

It is another object of the present invention to provide methods for the treatment of RNA-dependent RNA viral infection and in particular for the treatment of HCV infection.

It is another object of the present invention to provide methods for the treatment of RNA-dependent RNA viral infection in combination with other agents active against RNA-dependent RNA virus and in particular for the treatment of HCV infection in combination with other agents active against HCV.

It is another object of the present invention to provide fluorinated pyrrolo[2,3-d]pyrimidine nucleoside compounds and certain derivatives thereof and their pharmaceutical compositions for use as a medicament for the inhibition of RNA-dependent RNA viral replication and/or the treatment of RNA-dependent RNA viral infection and in particular for the inhibition of HCV replication and/or the treatment of HCV infection.

It is another object of the present invention to provide for the use of the fluorinated pyrrolo[2,3-d]pyrimidine nucleoside compounds and certain derivatives thereof of the present invention and their pharmaceutical compositions for the manufacture of a medicament for the inhibition of RNA-dependent RNA viral replication and/or the treatment of RNA-dependent RNA viral infection and in particular for the inhibition of HCV replication and/or the treatment of HCV infection.

These and other objects will become readily apparent from the detailed description which follows.

### SUMMARY OF THE INVENTION

The present invention relates to nucleoside compounds of structural formula I of the indicated stereochemical configuration:

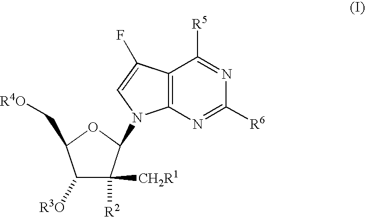

and pharmaceutically acceptable salts thereof; wherein
R¹ is hydrogen or fluorine;
R² is fluorine or hydroxy;
R³ is hydrogen, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, or an amino acyl residue of structural formula

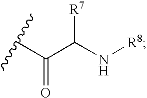

R⁴ is hydrogen, C₁₋₁₀ alkylcarbonyl, phosphoryl or a cyclic prodrug ester thereof, diphosphoryl, triphosphoryl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, an amino acyl residue of structural formula:

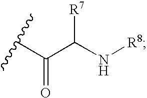

a residue of structural formula:

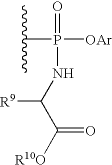

R⁵ is amino or hydroxy;
R⁶ is hydrogen, amino, or fluoro;
R⁷ is hydrogen, C₁₋₅ alkyl, or phenyl C₀₋₂ alkyl; and
R⁸ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl;
R⁹ is hydrogen, C₁₋₅ alkyl, phenyl or benzyl, wherein alkyl is unsubstituted or substituted with one substituent selected from the group consisting of hydroxy, methoxy, amino, carboxy, carbamoyl, guanidino, mercapto, methylthio, 1H-imidazolyl, and 1H-indol-3-yl and wherein phenyl and benzyl are unsubstituted or substituted with one to two substituents independently selected from the group consisting of halogen, hydroxy, and methoxy;
R¹⁰ is hydrogen, C₁₋₆ alkyl, C₃₋₆ cycloalkyl, phenyl, or benzyl, wherein alkyl and cycloalkyl are unsubstituted or substituted with one to three substituents independently selected from halogen, hydroxy, carboxy, C₁₋₄ alkoxy and wherein phenyl and benzyl are unsubstituted or substituted with one to three substituents independently selected from halogen, hydroxy, cyano, C₁₋₄ alkoxy, and trifluoromethyl; and
Ar is phenyl unsubstituted or substituted with one to three substituents independently selected from the group consisting of halogen, C₁₋₄ alkyl, C₁₋₄ alkoxy, C₁₋₄ alkylthio, cyano, nitro, amino, carboxy, trifluoromethyl, C₁₋₄ alkylamino, di(C₁₋₄ alkyl)amino, C₁₋₄ alkylcarbonyl, C₁₋₄ alkylcarbonyloxy, and C₁₋₄ alkyloxycarbonyl;
with the proviso that when R¹, R³, R⁴, and R⁶ are hydrogen and R² is hydroxy, then R⁵ cannot be amino.

The compounds of formula I are useful as inhibitors of RNA-dependent RNA viral polymerase and in particular of HCV NS5B polymerase. They are also inhibitors of RNA-dependent RNA viral replication and in particular of HCV replication and are useful for the treatment of RNA-dependent RNA viral infection and in particular for the treatment of HCV infection.

Also encompassed within the present invention are pharmaceutical compositions containing the compounds alone or in combination with other agents active against RNA-dependent RNA virus and in particular against HCV as well as methods for the inhibition of RNA-dependent RNA viral replication and for the treatment of RNA-dependent RNA viral infection.

### DETAILED DESCRIPTION OF THE INVENTION

The present invention relates to compounds of structural formula I of the indicated stereochemical configuration:

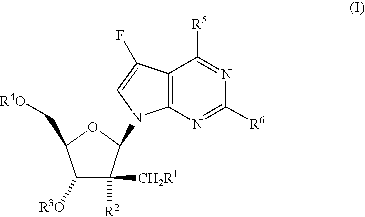

or a pharmaceutically acceptable salt thereof; wherein
R¹ is hydrogen or fluorine;
R² is fluorine or hydroxy;
R³ is hydrogen, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, or an amino acyl residue of structural formula

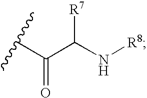

R⁴ is hydrogen, C₁₋₁₀ alkylcarbonyl, phosphoryl or a cyclic prodrug ester thereof, diphosphoryl, triphosphoryl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₁₄ alkyl)O(C═O)C₁₋₄ alkyl, an amino acyl residue of structural formula:

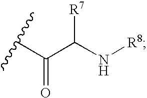

a residue of structural formula:

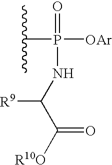

R⁵ is amino or hydroxy;
R⁶ is hydrogen, amino, or fluoro;
R⁷ is hydrogen, C₁₋₅ alkyl, or phenyl C₀₋₂ alkyl; and
R⁸ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl;
R⁹ is hydrogen, C₁₋₅ alkyl, phenyl or benzyl, wherein alkyl is unsubstituted or substituted with one substituent selected from the group consisting of hydroxy, methoxy, amino, carboxy, carbamoyl, guanidino, mercapto, methylthio, 1H-imidazolyl, and 1H-indol-3-yl and wherein phenyl and benzyl are unsubstituted or substituted with one to two substituents independently selected from the group consisting of halogen, hydroxy, and methoxy;
R¹⁰ is hydrogen, C₁₋₆ alkyl, C₃₋₆ cycloalkyl, phenyl, or benzyl, wherein alkyl and cycloalkyl are unsubstituted or substituted with one to three substituents independently selected from halogen, hydroxy, carboxy, C₁₋₄ alkoxy and wherein phenyl and benzyl are unsubstituted or substituted with one to three substituents independently selected from halogen, hydroxy, cyano, C₁₋₄ alkoxy, and trifluoromethyl; and
Ar is phenyl unsubstituted or substituted with one to three substituents independently selected from the group consisting of halogen, C₁₋₄ alkyl, C₁₋₄ alkoxy, C₁₋₄ alkylthio, cyano, nitro, amino, carboxy, trifluoromethyl, C₁₋₄ alkylamino, di(C₁₋₄ alkyl)amino, C₁₋₄ alkylcarbonyl, C₁₋₄ alkylcarbonyloxy, and C₁₋₄ alkyloxycarbonyl;
with the proviso that when R¹, R³, R⁴, and R⁶ are hydrogen and R² is hydroxy, then R⁵ cannot be amino.

The compounds of formula I are useful as inhibitors of RNA-dependent RNA viral polymerase. They are also inhibitors of RNA-dependent RNA viral replication and are useful for the treatment of RNA-dependent RNA viral infection.

In one embodiment of the compounds of the present invention, R¹ is hydrogen; R² is hydroxy; and R³ and R⁴ are hydrogen.

In a second embodiment of the compounds of the present invention, R¹ is hydrogen; R² is fluoro; and R³ and R⁴ are hydrogen; with the proviso that when R¹, R³, R⁴, and R⁶ are hydrogen and R² is hydroxy, then R⁵ cannot be amino.

In a third embodiment of the compounds of the present invention, Ar is unsubstituted phenyl.

In a fourth embodiment of the compounds of the present invention, R⁹ is selected from the group consisting of hydrogen, methyl, ethyl, n-propyl, isopropyl, isobutyl, 2-methyl-1-propyl, hydroxymethyl, mercaptomethyl, carboxymethyl, carbamoylmethyl, 1-hydroxyethyl, 2-carboxyethyl, 2-carbamoylethyl, 2-methylthioethyl, 4-amino-1-butyl, 3-amino-1-propyl, 3-guanidino-1-propyl, 1H-imidazol-4-ylmethyl, phenyl, 4-hydroxybenzyl, and 1H-indol-3-ylmethyl. In a class of this embodiment, R⁹ is methyl or benzyl.

In a fifth embodiment of the compounds of the present invention, R¹⁰ is C₁₋₆ alkyl, cyclohexyl, phenyl or benzyl. In a class of this embodiment, R¹⁰ is methyl.

In a sixth embodiment of the compounds of the present invention, Ar is unsubstituted phenyl, R⁹ is methyl or benzyl, and R¹⁰ is methyl.

Illustrative, but nonlimiting, examples of compounds of the present invention of structural formula I which are useful as inhibitors of RNA-dependent RNA viral polymerase are the following:

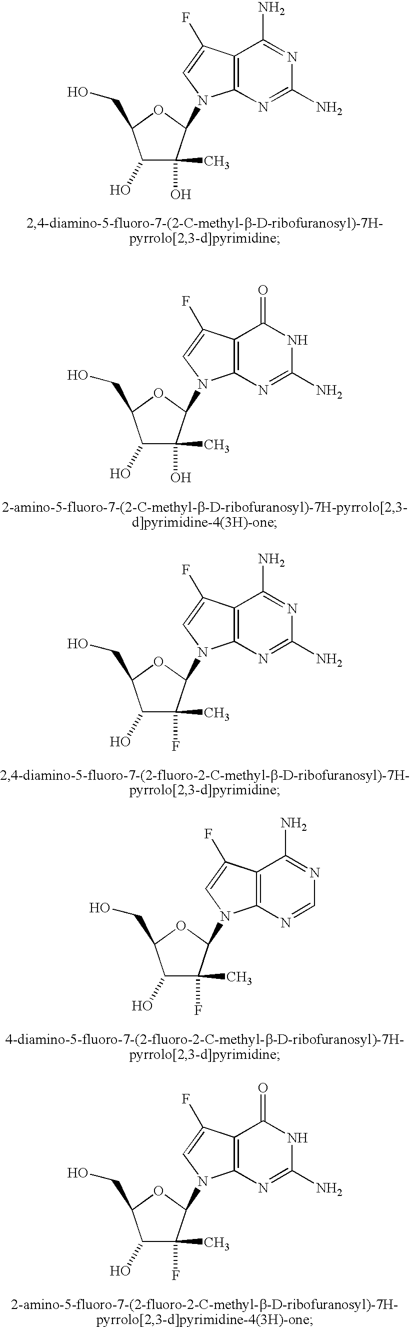

and pharmaceutically acceptable salts thereof.

In one embodiment of the present invention, the fluorinated pyrrolo[2,3-d]pyrimidine nucleoside compounds of the present invention are useful as inhibitors of positive-sense single-stranded RNA-dependent RNA viral polymerase, inhibitors of positive-sense single-stranded RNA-dependent RNA viral replication, and/or for the treatment of positive-sense single-stranded RNA-dependent RNA viral infection. In a class of this embodiment, the positive-sense single-stranded RNA-dependent RNA virus is a Flaviviridae virus or a Picornaviridae virus. In a subclass of this class, the Picornaviridae virus is a rhinovirus, a poliovirus, or a hepatitis A virus. In a second subclass of this class, the Flaviviridae virus is selected from the group consisting of hepatitis C virus, yellow fever virus, dengue virus, West Nile virus, Japanese encephalitis virus, Banzi virus, and bovine viral diarrhea virus (BVDV). In a subclass of this subclass, the Flaviviridae virus is hepatitis C virus.

Another aspect of the present invention is concerned with a method for inhibiting RNA-dependent RNA viral polymerase, a method for inhibiting RNA-dependent RNA viral replication, and/or a method for treating RNA-dependent RNA viral infection in a mammal in need thereof comprising administering to the mammal a therapeutically effective amount of a compound of structural formula I.

In one embodiment of this aspect of the present invention, the RNA-dependent RNA viral polymerase is a positive-sense single-stranded RNA-dependent RNA viral polymerase. In a class of this embodiment, the positive-sense single-stranded RNA-dependent RNA viral polymerase is a Flaviviridae viral polymerase or a Picornaviridae viral polymerase. In a subclass of this class, the Picornaviridae viral polymerase is rhinovirus polymerase, poliovirus polymerase, or hepatitis A virus polymerase. In a second subclass of this class, the Flaviviridae viral polymerase is selected from the group consisting of hepatitis C virus polymerase, yellow fever virus polymerase, dengue virus polymerase, West Nile virus polymerase, Japanese encephalitis virus polymerase, Banzi virus polymerase, and bovine viral diarrhea virus (BVDV) polymerase. In a subclass of this subclass, the Flaviviridae viral polymerase is hepatitis C virus polymerase.

In a second embodiment of this aspect of the present invention, the RNA-dependent RNA viral replication is a positive-sense single-stranded RNA-dependent RNA viral replication. In a class of this embodiment, the positive-sense single-stranded RNA-dependent RNA viral replication is Flaviviridae viral replication or Picornaviridae viral replication. In a subclass of this class, the Picornaviridae viral replication is rhinovirus replication, poliovirus replication, or hepatitis A virus replication. In a second subclass of this class, the Flaviviridae viral replication is selected from the group consisting of hepatitis C virus replication, yellow fever virus replication, dengue virus replication, West Nile virus replication, Japanese encephalitis virus replication, Banzi virus replication, and bovine viral diarrhea virus replication. In a subclass of this subclass, the Flaviviridae viral replication is hepatitis C virus replication.

In a third embodiment of this aspect of the present invention, the RNA-dependent RNA viral infection is a positive-sense single-stranded RNA-dependent viral infection. In a class of this embodiment, the positive-sense single-stranded RNA-dependent RNA viral infection is Flaviviridae viral infection or Picornaviridae viral infection. In a subclass of this class, the Picornaviridae viral infection is rhinovirus infection, poliovirus infection, or hepatitis A virus infection. In a second subclass of this class, the Flaviviridae viral infection is selected from the group consisting of hepatitis C virus infection, yellow fever virus infection, dengue virus infection, West Nile virus infection, Japanese encephalitis virus infection, Banzi virus infection, and bovine viral diarrhea virus infection. In a subclass of this subclass, the Flaviviridae viral infection is hepatitis C virus infection.

Throughout the instant application, the following terms have the indicated meanings:

“Alkyl”, as well as other groups having the prefix “alk”, such as alkoxy and alkylthio, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” shall mean straight or branched chain alkenes of two to six total carbon atoms, or any number within this range (e.g., ethenyl, propenyl, butenyl, pentenyl, etc.).

The term “alkynyl” shall mean straight or branched chain alkynes of two to six total carbon atoms, or any number within this range (e.g., ethynyl, propynyl, butynyl, pentynyl, etc.).

The term “alkoxy” refers to straight or branched chain alkoxides of the number of carbon atoms specified (e.g., C₁₋₄ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₄ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₄ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkyloxycarbonyl” refers to straight or branched chain esters of a carboxylic acid derivative of the present invention of the number of carbon atoms specified (e.g., C₁₋₄ alkyloxycarbonyl), or any number within this range [i.e., methyloxycarbonyl (MeOCO—), ethyloxycarbonyl, or butyloxycarbonyl].

The term “alkylcarbonyl” refers to straight or branched chain alkylacyl group of the number of carbon atoms specified (e.g., C₁₋₄ alkylcarbonyl), or any number within this range [i.e., methylcarbonyl (MeCO—), ethylcarbonyl, or butylcarbonyl].

The term “halogen” is intended to include the halogen atoms fluorine, chlorine, bromine and iodine.

The term “phosphoryl” refers to —P(O)(OH)₂.

The term “diphosphoryl” refers to the radical having the structure:

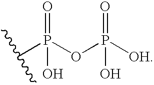

The term “triphosphoryl” refers to the radical having the structure:

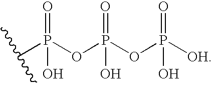

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally.

When R⁷ in the amino acyl residue embodiment of R³ and R⁴ is other than hydrogen in the formula

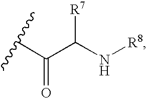

the amino acyl residue contains an asymmetric center and is intended to include the individual R- and S-stereoisomers as well as RS-diastereoisomeric mixtures.

The term “5′-triphosphate” refers to a triphosphoric acid ester derivative of the 5′-hydroxyl group of a fluorinated pyrrolo[2,3-d]pyrimidine nucleoside compound of the present invention having the following general structural formula II:

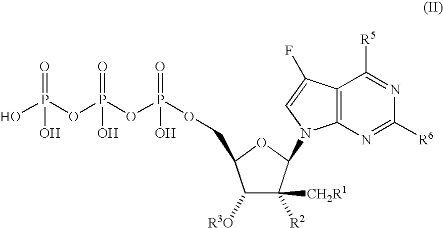

wherein R¹-R³, R⁵, and R⁶ are as defined above. The compounds of the present invention are also intended to include pharmaceutically acceptable salts of the triphosphate ester as well as pharmaceutically acceptable salts of 5′-monophosphate and 5′-diphosphate ester derivatives of the structural formulae III and IV, respectively,

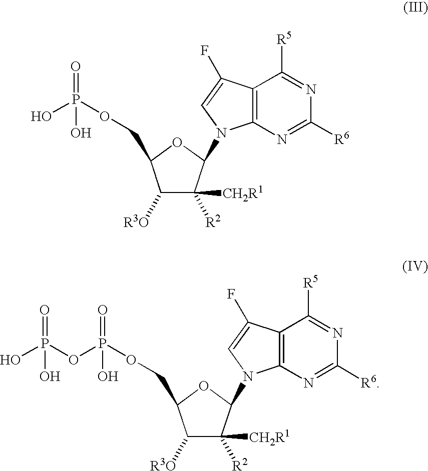

The term “composition”, as in “pharmaceutical composition,” is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “administration of” and “administering a” compound should be understood to mean providing a compound of the invention or a prodrug of a compound of the invention to the individual in need.

Another aspect of the present invention is concerned with a method of inhibiting HCV NS5B polymerase, inhibiting HCV replication, or treating HCV infection with a compound of the present invention in combination with one or more agents useful for treating HCV infection. Such agents active against HCV include, but are not limited to, ribavirin, levovirin, viramidine, thymosin alpha-1, interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), pegylated interferon-α2a (Pegasys™), interferon-α2b (such as Intron-A interferon available from Schering Corp., Kenilworth, N.J.), pegylated interferon-α2b (PegIntron™), a recombinant consensus interferon (such as interferon alphacon-1), and a purified interferon-α product. Amgen's recombinant consensus interferon has the brand name Infergen®. Levovirin is the L-enantiomer of ribavirin which has shown immunomodulatory activity similar to ribavirin. Viramidine represents an analog of ribavirin disclosed in WO 01/60379 (assigned to ICN Pharmaceuticals). In accordance with this method of the present invention, the individual components of the combination can be administered separately at different times during the course of therapy or concurrently in divided or single combination forms. The instant invention is therefore to be understood as embracing all such regimes of simultaneous or alternating treatment, and the term “administering” is to be interpreted accordingly. It will be understood that the scope of combinations of the compounds of this invention with other agents useful for treating HCV infection includes in principle any combination with any pharmaceutical composition for treating HCV infection. When a compound of the present invention or a pharmaceutically acceptable salt thereof is used in combination with a second therapeutic agent active against HCV, the dose of each compound may be either the same as or different from the dose when the compound is used alone.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Both substrate and non-substrate based inhibitors of HCV NS3 protease inhibitors are disclosed in WO 98/22496, WO 98/46630, WO 99/07733, WO 99/07734, WO 99/38888, WO 99/50230, WO 99/64442, WO 00/09543, WO 00/59929, GB-2337262, WO 02/48116, WO 02/48172, and U.S. Pat. No. 6,323,180. HCV NS3 protease as a target for the development of inhibitors of HCV replication and for the treatment of HCV infection is discussed in B. W. Dymock, “Emerging therapies for hepatitis C virus infection,” Emerging Drugs, 6: 1342 (2001).

Ribavirin, levovirin, and viramidine may exert their anti-HCV effects by modulating intracellular pools of guanine nucleotides via inhibition of the intracellular enzyme inosine monophosphate dehydrogenase (IMPDH). IMPDH is the rate-limiting enzyme on the biosynthetic route in de novo guanine nucleotide biosynthesis. Ribavirin is readily phosphorylated intracellularly and the monophosphate derivative is an inhibitor of IMPDH. Thus, inhibition of IMPDH represents another useful target for the discovery of inhibitors of HCV replication. Therefore, the compounds of the present invention may also be administered in combination with an inhibitor of IMPDH, such as VX-497, which is disclosed in WO 97/41211 and WO 01/00622 (assigned to Vertex); another IMPDH inhibitor, such as that disclosed in WO 00/25780 (assigned to Bristol-Myers Squibb); or mycophenolate mofetil [see A. C. Allison and E. M. Eugui, Agents Action, 44 (Suppl.): 165 (1993)].

For the treatment of HCV infection, the compounds of the present invention may also be administered in combination with the antiviral agent amantadine (1-aminoadamantane) [for a comprehensive description of this agent, see J. Kirschbaum, Anal. Profiles Drug Subs. 12: 1-36 (1983)].

The compounds of the present invention may also be combined for the treatment of HCV infection with antiviral 2′-C-branched ribonucleosides disclosed in R. E. Harry-O'kuru, et al., J. Org. Chem., 62: 1754-1759 (1997); M. S. Wolfe, et al., Tetrahedron Lett., 36: 7611-7614 (1995); U.S. Pat. No. 3,480,613 (Nov. 25, 1969); International Publication Number WO 01/90121 (29 Nov. 2001); International Publication Number WO 01/92282 (6 Dec. 2001); and International Publication Number WO 02/32920 (25 Apr. 2002); and International Publication Number WO 04/002999 (8 Jan. 2004); and International Publication Number WO 04/003000 (8 Jan. 2004); and International Publication Number WO 04/002422 (8 Jan. 2004); the contents of each of which are incorporated by reference in their entirety. Such 2′-C-branched ribonucleosides include, but are not limited to, 2′-C-methylcytidine, 2′-C-methyluridine, 2′-C-methyladenosine, 2′-C-methylguanosine, and 9-(2-C-methyl-β-D-ribofuranosyl)-2,6-diaminopurine, and the corresponding amino acid ester of the ribose C-2′, C-3′, and C-5′ hydroxyls (such as, 3′-O-(L-valyl)-2′-C-methylcytidine) and the corresponding optionally substituted cyclic 1,3-propanediol esters of the 5′-phosphate derivatives.

The compounds of the present invention may also be combined for the treatment of HCV infection with other nucleosides having anti-HCV properties, such as those disclosed in WO 02/51425 (4 Jul. 2002), assigned to Mitsubishi Pharma Corp.; WO 01/79246, WO 02/32920, and WO 02/48165 (20 Jun. 2002), assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003): US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004).

The compounds of the present invention may also be combined for the treatment of HCV infection with non-nucleoside inhibitors of HCV polymerase such as those disclosed in WO 01/77091 (18 Oct. 2001), assigned to Tularik, Inc.; WO 01/47883 (5 Jul. 2001), assigned to Japan Tobacco, Inc.; WO 02/04425 (17 Jan. 2002), assigned to Boehringer Ingelheim; WO 02/06246 (24 Jan. 2002), assigned to Istituto di Ricerche di Biologia Moleculare P. Angeletti S.P.A.; and WO 02/20497 (3 Mar. 2002).

By “pharmaceutically acceptable” is meant that the carrier, diluent, or excipient must be compatible with the other ingredients of the formulation and not deleterious to the recipient thereof.

Also included within the present invention are pharmaceutical compositions comprising the fluorinated pyrrolo[2,3-d]pyrimidine nucleoside compounds and derivatives thereof of the present invention in association with a pharmaceutically acceptable carrier. Another example of the invention is a pharmaceutical composition made by combining any of the compounds described above and a pharmaceutically acceptable carrier. Another illustration of the invention is a process for making a pharmaceutical composition comprising combining any of the compounds described above and a pharmaceutically acceptable carrier.

Also included within the present invention are pharmaceutical compositions useful for inhibiting RNA-dependent RNA viral polymerase in particular HCV NS5B polymerase comprising an effective amount of a compound of the present invention and a pharmaceutically acceptable carrier. Pharmaceutical compositions useful for treating RNA-dependent RNA viral infection in particular HCV infection are also encompassed by the present invention as well as a method of inhibiting RNA-dependent RNA viral polymerase in particular HCV NS5B polymerase and a method of treating RNA-dependent viral replication and in particular HCV replication. Additionally, the present invention is directed to a pharmaceutical composition comprising a therapeutically effective amount of a compound of the present invention in combination with a therapeutically effective amount of another agent active against RNA-dependent RNA virus and in particular against HCV. Agents active against HCV include, but are not limited to, ribavirin, levovirin, viramidine, thymosin alpha-1, an inhibitor of HCV NS3 serine protease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as Roferon interferon available from Hoffmann-LaRoche, Nutley, N.J.), interferon-α2b (such as Intron-A interferon available from Schering Corp., Kenilworth, N.J.), a consensus interferon, and a purified interferon-1 product. For a discussion of ribavirin and its activity against HCV, see J. O. Saunders and S. A. Raybuck, “Inosine Monophosphate Dehydrogenase: Consideration of Structure, Kinetics, and Therapeutic Potential,” Ann. Rep. Med. Chem., 35: 201-210 (2000).

Another aspect of the present invention provides for the use of the fluorinated pyrrolo[2,3-d]pyrimidine nucleoside compounds and derivatives thereof and their pharmaceutical compositions for the manufacture of a medicament for the inhibition of RNA-dependent RNA viral replication, in particular HCV replication, and/or the treatment of RNA-dependent RNA viral infection, in particular HCV infection. Yet a further aspect of the present invention provides for the fluorinated pyrrolo[2,3-d]pyrimidine nucleoside compounds and derivatives thereof and their pharmaceutical compositions for use as a medicament for the inhibition of RNA-dependent RNA viral replication, in particular HCV replication, and/or for the treatment of RNA-dependent RNA viral infection, in particular HCV infection.

The pharmaceutical compositions of the present invention comprise a compound of structural formula I as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of structural formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of structural formula I may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of structural formula I are administered orally.

For oral administration to humans, the dosage range is 0.01 to 1000 mg/kg body weight in divided doses. In one embodiment the dosage range is 0.1 to 100 mg/kg body weight in divided doses. In another embodiment the dosage range is 0.5 to 20 mg/kg body weight in divided doses. For oral administration, the compositions are preferably provided in the form of tablets or capsules containing 1.0 to 1000 milligrams of the active ingredient, particularly, 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated.

The effective dosage of active ingredient employed may vary depending on the particular compound employed, the mode of administration, the condition being treated and the severity of the condition being treated. Such dosage may be ascertained readily by a person skilled in the art. This dosage regimen may be adjusted to provide the optimal therapeutic response.

The compounds of the present invention contain one or more asymmetric centers and can thus occur as racemates and racemic mixtures, single enantiomers, diastereomeric mixtures and individual diastereomers. The present invention is meant to comprehend fluorinated pyrrolo[2,3-d]pyrimidine nucleoside compounds having the β-D stereochemical configuration for the five-membered furanose ring as depicted in the structural formula below, that is, fluorinated pyrrolo[2,3-d]pyrimidine nucleoside compounds in which the substituents at C-1 and C-4 of the five-membered furanose ring have the β-stereochemical configuration (“up” orientation as denoted by a bold line).

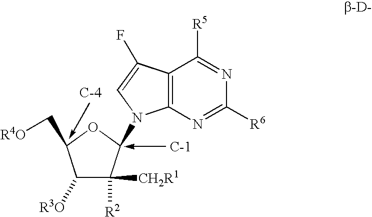

Some of the compounds described herein contain olefinic double bonds, and unless specified otherwise, are meant to include both E and Z geometric isomers.

Some of the compounds described herein may exist as tautomers such as keto-enol and imine-enamine tautomers. The individual tautomers as well as mixtures thereof are encompassed with compounds of structural formula I. Example of keto-enol and imine-enamine tautomers which are intended to be encompassed within the compounds of the present invention are illustrated below:

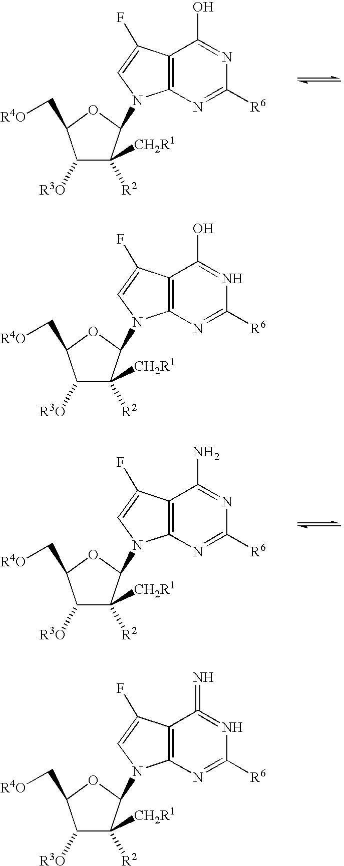

Compounds of structural formula I may be separated into their individual diastereoisomers by, for example, fractional crystallization from a suitable solvent, for example methanol or ethyl acetate or a mixture thereof, or via chiral chromatography using an optically active stationary phase.

Alternatively, any stereoisomer of a compound of the structural formula I may be obtained by stereospecific synthesis using optically pure starting materials or reagents of known configuration.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH), phosphoric acid [—OP(O)(OH)₂], or alcohol group being present in the compounds of the present invention, pharmaceutically acceptable prodrug esters of carboxylic acid derivatives, such as methyl, ethyl, or pivaloyloxymethyl esters; pharmaceutically acceptable prodrug esters of 5′-phosphoric acid derivatives (including 5′-monophosphate, 5′-diphosphate, and 5′-triphosphate) of the fluorinated pyrrolo[2,3-d]pyrimidine nucleoside; or prodrug acyl derivatives of the ribose C-2′, C-3′, and C-5′ hydroxyls, such as O-acetate, O-maleate, and O-aminoacyl, can be employed. Included are those esters and acyl groups known in the art for modifying the bioavailability, tissue distribution, solubility, and hydrolysis characteristics for use as sustained-release or prodrug formulations. Also included are five-membered cyclic carbonate derivatives of the C-2′ and C-3′ hydroxyls. The contemplated derivatives are readily convertible in vivo into the required compound. Thus, in the methods of treatment of the present invention, the terms “administering” and “administration” is meant to encompass the treatment of the viral infections described with a compound specifically disclosed or with a compound which may not be specifically disclosed, but which converts to the specified compound in vivo after administration to the mammal, including a human patient. Conventional procedures for the selection and preparation of suitable prodrug derivatives are described, for example, in “Design of Prodrugs,” ed. H. Bundgaard, Elsevier, 1985, which is incorporated by reference herein in its entirety.

### Preparation of the Compounds of the Invention:

A starting material for the preparation of the compounds of the present invention is 4-amino-5-fluoro-7-(2-C-methyl-β-D-ribofuranosyl)-7H-pyrrolo[2,3-d]pyrimidine (1-9) whose synthesis is depicted in Scheme 1.

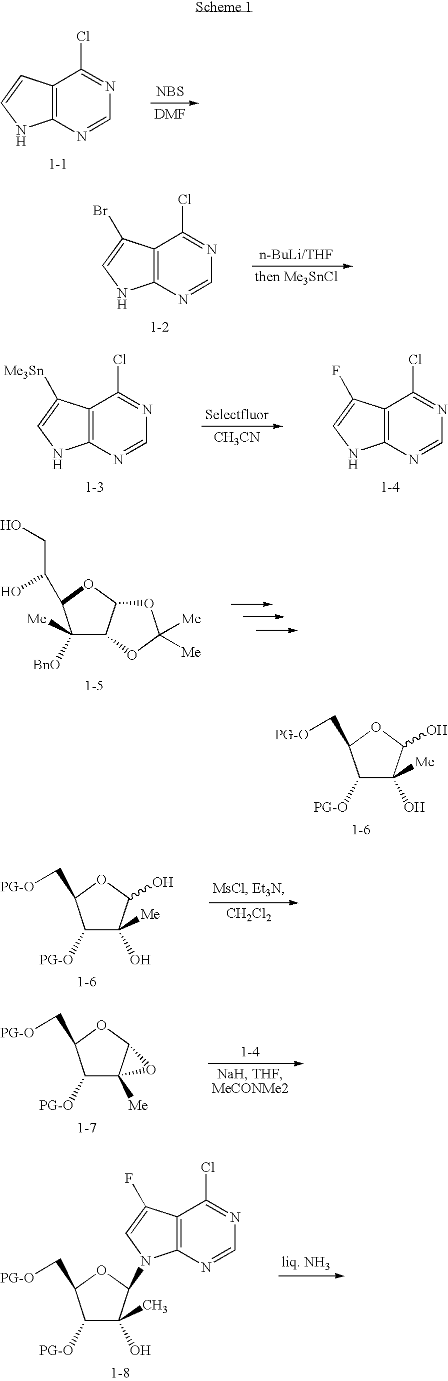

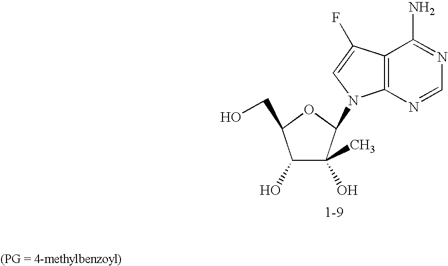

### Preparation of 5-fluoro-4-chloro-7H-pyrrolo[2,3-d]pyrimidine (1-4)

### Step A: 5-Bromo-4-chloro-7H-pyrrolo[2,3-d]pyrimidine (1-2)

To a solution of 4-chloro-7H-pyrrolo[2,3-d]pyrimidine (1-1) (1.53 g, 10.0 mmol) in DMF (20 mL) was added N-bromosuccinimide (1.78 g, 10.0 mmol) in DMF (10 mL) dropwise at 0° C. The reaction mixture was stirred at 0° C. for 30 min and then at room temperature for 1 h. Methanol (25 mL) was added, and the reaction mixture was stirred for an additional 1 h. The solvent was evaporated and the residue was crystallized from methanol to give the title compound as white solid.

### Step B: 5-(Trimethylstannyl)-4-chloro-7H-pyrrolo[2,3-d]pyrimidine (1-3)

To a solution of the compound from Step A (0.92 g, 4 mmol) in THF (25 mL) was added n-BuLi (2.5 M solution in hexane, 3.48 mL) dropwise at −78° C. After the addition, the reaction mixture was stirred at −78° C. for an additional 30 min. To this solution was added trimethyltin chloride (0.88 g, 4.4 mmol) in THF (8 mL) dropwise for a period of 10 min. The reaction mixture was brought to room temperature slowly and stirred at room temperature overnight. Saturated aqueous ammonium chloride (60 mL) was added and extracted with ethyl acetate (3×70 mL). The combined organic extracts were washed with brine, dried over Na₂SO₄ and evaporated to dryness. The residue was purified over silica gel to give the title compound as a colorless solid.

### Step C: 5-Fluoro-4-chloro-7H-pyrrolo[2,3-d]pyrimidine (1-4)

To a solution of the compound from Step B (1.97 g, 6.20 mmol) in CH₃CN (60 mL) was added [1-(chloromethyl)-4-fluoro-1,4-diazoniabicyclo[2.2.2]octane bis(tetrafluoroborate)] (SELECTFLUOR® fluorinating reagent) (2.40 g, 6.5 mmol) in one portion and the reaction mixture was stirred at room temperature for 7 h. The white precipitate was filtered off, and the filtrate was evaporated to dryness. The residue was purified over silica gel using ethyl acetate/hexane (3:7) as the eluent. Fractions containing the product were pooled and evaporated in vacuo to give the title compound as a colorless solid.

¹H-NMR (500 MHz, MeOH-d₄): δ 8.53 (s, 1H), 7.37 (d, J=2.8 Hz); ¹⁹F-NMR (DMSO-d₆): δ −171.5.

### Preparation of 2-C-methyl-3,5-di-O-(p-toluoyl)-D-ribofuranose (1-6)

To a solution of 3-O-benzyl-1,2-O-isopropylidene-3-C-methyl-α-D-allofuranose (L-5) (for preparation, see Carbohydr. Res., 44: 275-283 (1975) (5.0 kg, 15.4 mol) and pyridine (3.7 kg, 46.2 mol) in 35 L of acetonitrile was added p-toluoyl chloride (5.2 kg, 33.9 mol), and the reaction was heated at 50-55° C. for 12 h. A solution of 6.0 L (46.2 mol) of 48 wt % HBF₄ (tetrafluoroboric acid) in 9 L of water was added at 50-55° C. After 2 h, 10 L of acetonitrile was distilled off, and 10 L acetonitrile was added. At 97% conversion, 10 L of acetonitrile was distilled off, and the reaction solution was cooled to 0-5° C. A solution of periodic acid (4.2 kg, 18.5 mol) in 10 L of water was added. After the reaction was aged for 30 min, 35 L of isopropyl acetate and 10 L of water were added. The organic phase was washed with 25 L of water followed by 20 L of aqueous NaHCO₃, 15 L of 5% sodium thiosulfate in water, and 15 L of water. The isopropyl acetate solution was concentrated to 10-15 L, and 40 L of methanol was added. The solution was cooled to 0° C. and diisopropylamine (0.78 kg, 7.7 mol) was added. After 2 d at 0° C., aqueous HCl (1N, 7.7 L) was added at 0-5° C. followed by 30 L of isopropyl acetate and 40 L of water. The organic phase was washed with aqueous 1N HCl, NaHCO₃, and brine. The organic phase was dried through azeotropic distillation and treated with activated carbon. The carbon was removed by filtration and the resulting solution was diluted to 75 L with isopropyl acetate and hydrogenated (45 psi, 50° C., 1.5 kg 10% Pd/C) for 24 h. The filtrate was concentrated to 15 L and 60 L of heptane was added at 50° C. The crystalline product was isolated by filtration washing with a 10 L of 20% isopropyl acetate in heptane. Drying afforded 4.03 kg of the desired diol 1-6.

¹H NMR (CDCl₃, 400 MHz): The ratio of α:β isomers in CDCl₃ is about 5 to 1. For the major isomer: δ7.95-7.90 (m, 4H), 7.26 (d, J=8.0 Hz, 2H), 7.17 (d, J=8.0 Hz, 2H), 5.53 (d, J=7.2 Hz, 1H), 5.22 (d, J=2.8 Hz, 1H), 4.65-4.49 (m, 3H), 3.08 (d, J=3.2 Hz, 1H), 2.44 (s, 3H), 2.38 (s, 3H), 2.26 (s, 1H), 1.44 (s, 3H) ppm; for the minor isomer: δ 7.95-7.90 (m, 4H), 7.27 (d, J=8.0 Hz, 2H), 7.22 (d, J=8.0 Hz, 2H), 5.16 (d, J=5.6 Hz, 1H), 5.12 (d, J=5.6 Hz, 1H), 4.66-4.49 (m, 3H), 3.54 (d, J=5.6 Hz, 1H), 2.91 (s, 1H), 2.43 (s, 3H), 2.40 (s, 3H), 1.44 (s, 3H) ppm.

### Preparation of 4-amino-5-fluoro-7-(2-C-methyl-β-D-ribofuranosyl)-7H)-pyrrolo[2,3-d]pyridine (1-9)

### Step A: 1,2-Anhydro-3,5-di-O-(p-toluoyl)-2-C-methyl-α-D-ribofuranose (1-7)

To a 72 L vessel was charged dry dichloromethane (32 L), triethylamine (3.0 L), and diol 2-2 (3.44 kg, 90 wt % pure). The mixture was warmed to 30° C., then methanesulfonyl chloride (0.79 L) was added over 40 min. After 1 h, the batch was partitioned between pH 7 buffer (20 L) and methyl tert-butyl ether (44 L). The organic phase was washed with 1M aqueous NaCl (38 L) then switched to toluene by vacuum distillation followed by concentration to about 9 L. The resulting solution of epoxide was used directly in Step B.

### Step B: 4-Chloro-5-fluoro-7-(2-C-methyl-3,5-di-O-(p-toluoyl)-β-D-ribofuranosyl)-7H-pyrrolo[2,3-d]pyrimidine (1-8)

To a solution of 4-chloro-5-fluoro-7H-pyrrolo[2,3-d]pyrimidine (1-4) (28.4 g, 0.165 mol) in N,N-dimethylacetamide (300 mL) was added portionwise 60% sodium hydride (6.6 g, 0.165 mol) at room temperature. After the addition, the reaction mixture stirred at 60° C. for one h. To the reaction mixture was added a solution of 1,2-anhydro-3,5-di-O-(p-toluoyl)-2-C-methyl-α-D-ribofuranose (1-7) (63.4 g, 0.166 mol) in THF (200 mL) and the reaction mixture was heated at 60° C. for 18 h. The reaction mixture was cooled to room temperature and poured into water (1 L) and ethyl acetate (2 L). The organic extract was washed with water (500 mL), dried over MgSO₄, and evaporated to dryness. The residue was purified over silica gel using 10-40% ethyl acetate/hexane as the eluant. Fractions containing the product were combined and concentrated to a foam which was used directly in Step C below.

### Step C: 4-Amino-5-fluoro-7-(2-C-methyl-β-D-ribofuranosyl)-7H-pyrrolo[2,3-d]pyrimidine (1-9)

A solution of 4-chloro-5-fluoro-7-(2-C-methyl-3,5-di-O-(p-toluoyl)-β-D-ribofuranosyl)-7H-pyrrolo[2,3-d]pyrimidine (1-8) (29.4 g, 0.053 mol) in anhydrous ammonia (300 mL) was heated at 85° C. in a sealed vessel for 48 h. The reaction mixture warmed to room temperature and the residue was slurried in methanol (200 mL), filtered, and the filtrate was adsorbed onto silica gel (200 g) then purified by chromatography using 0-30% methanol/methylene chloride as the eluant. Fractions containing product were combined and evaporated to give the title compound 1-9 as a solid.

¹H-NMR (500 MHz, MeOH-d₄): δ 8.07 (s, 1H), 7.41 (d, J=2.2 Hz, 1H), 6.25 (d, J=1.8 Hz), 4.09-3.95 (m, 3H), 3.82 (dd, J=2.7, 12.5 Hz, 1H); ¹⁹F-NMR (MeOH-d₄): δ −170.4; mass spectrum: 321 (M+Na)⁺.

Introduction of an amino group at the C-2 position of the 6-amino-7-deazapurine (7-deazaadenine) ring in intermediate 1-9 was carried out following synthetic methods described by H. Zhao, et al., in J. Org. Chem., 62: 7832-7835 (1997), as exemplified in Scheme 2. The 2,6-diamino-7-deazapurine ring can be converted into a 7-deazaguanine system following methods described by K. Alarcon et al., in Tetrahedron Lett., 41: 7211-7215 (2000), as exemplified in Scheme 3.

The Examples below provide illustrations of the conditions used for the preparation of the compounds of the present invention. These Examples are not intended to be limitations on the scope of the instant invention in any way, and they should not be so construed. Those skilled in the art of nucleoside and nucleotide synthesis will readily appreciate that known variations of the conditions and processes of the following preparative procedures can be used to prepare these and other compounds of the present invention. All temperatures are degrees Celsius unless otherwise noted.

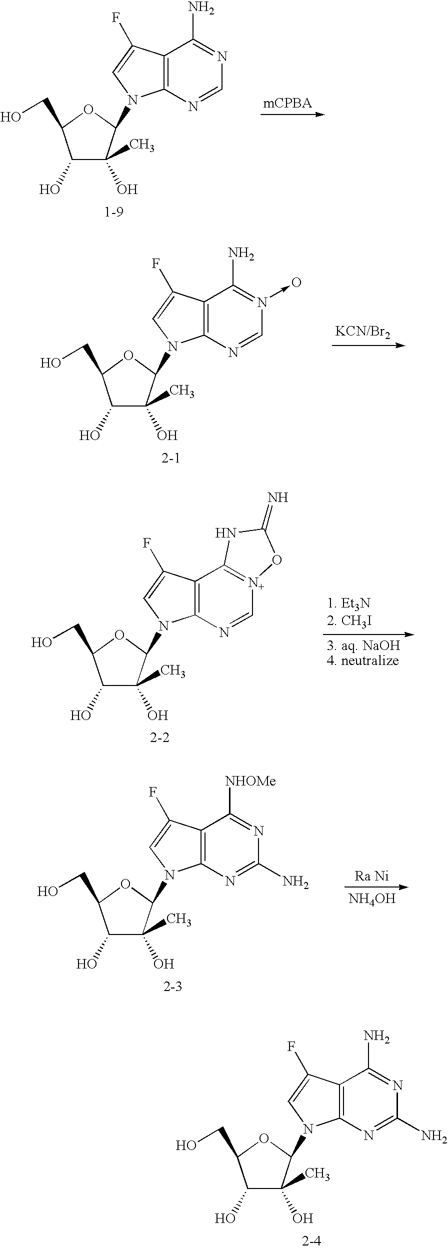

### EXAMPLE 1

### 2,4-Diamino-5-fluoro-7-(2-C-methyl-β-D-ribofuranosyl)-7H-pyrrolo[2,3-d]pyrimidine (2-4)

### Step A: 4-Amino-5-fluoro-3-N-oxo-7-(2-C-methyl-β-D-ribofuranosyl)-7H-pyrrolo[2,3-d]pyrimidine (2-1)

To a solution of 4-amino-5-fluoro-7-(2-C-methyl-β-D-ribofuranosyl)-7H-pyrrolo[2,3-d]pyrimidine (1-9) (268 mg, 0.899 mmol) in 50% methanol/water (20 mL) was added m-chloroperoxybenzoic acid (444 mg, 1.80 mmol). The reaction mixture stirred at room temperature for 18 h. The solvent was evaporated and the residue was azeotroped two times with toluene to give the title compound as a beige solid.

### Step B: 2,4-Diamino-5-fluoro-7-(2-C-methyl-β-ribofuranosyl)-7H-pyrrolo[2,3-d]pyrimidine (2-4)

To a solution of cyanogen bromide (75 mg, 0.708 mmol) in water (3 mL) was added a solution of 4-amino-5-fluoro-3-N-oxo-7-(2-C-methyl-β-D-ribofuranosyl)-7H-pyrrolo[2,3-d]pyrimidine (2-1) (160 mg, 0.509 mmol) in water (3 mL) at 0° C. The resulting solution stirred at 0° C. for 1.5 h. The solvent was evaporated and azeotroped with toluene. To the residue was added N,N-dimethylformamide (3.5 mL) and triethylamine (0.25 mL, 1.79 mol) and the resulting solution stirred at room temperature for 45 min. Iodomethane (0.25 mL, 4.0 mmol) was added portionwise and the reaction mixture stirred at room temperature in darkness for 1.5 h. The solvent was evaporated and 0.25M aqueous sodium hydroxide (10 mL) was added to the residue which was stirred at room temperature for 30 min. The reaction mixture was neutralized with 1M HCl, diluted with ethanol (10 mL) and heated at 60° C. for 8 h. After cooling to room temperature, concentrated ammonium hydroxide (12 mL) was added and the reaction mixture was heated to 90° C. whereupon Raney nickel was added. After 15 min, the hot reaction mixture was filtered through solka-floc, the solvent was evaporated and the residue was purified over silica gel using methanol/methylene chloride as the eluant. Fractions containing the product were evaporated to give the title compound 2-4 as a solid.

¹H-NMR (500 MHz, MeOH-d₄): δ 7.3 (s, 1H), 6.1 (s, 1H), 4.0 (m, 3H), 3.8 (d, 1H), 0.9 (s, 3H); Mass spectrum: 314 (M+1).

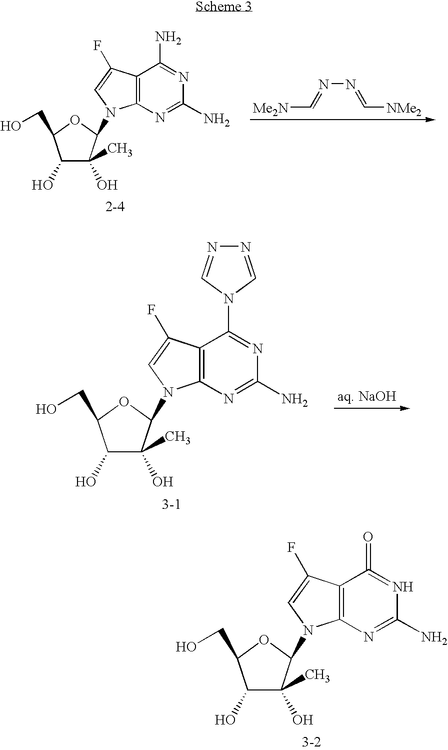

### EXAMPLE 2

### 2-amino-5-fluoro-7-(2-C-methyl-β-D-ribofuranosyl)-7H-pyrrolo[2,3-d]pyrimidin-4(3H)-one (3-2)

This compound is prepared by treating compound 2-4 with 1,2-bis[(dimethylamino)-methylene]hydrazine in DMF to afford triazole 3-1 which is hydrolyzed with 1N aqueous NaOH in DMSO following the conditions described by K. Alarcon et al., in Tetrahedron Lett., 41: 7211-7215 (2000).

The 2-fluoro-2-C-methylribonucleosides (R²=F in structural formula I) of the present invention are prepared following synthetic methodologies well-established in the practice of nucleoside and nucleotide chemistry. As an illustration is provided the preparation of compound 6-3 (Example 3) as depicted in Schemes 4-6. D-Ribose (4-1) is first protected. In this case, an ester, such as acetate and benzoate, presents a suitable protecting group, but alternative protecting groups may be used as well. The esterification is achieved by reacting D-ribose with the appropriate acyl halide or anhydride optionally in the presence of a solvent, such as diethyl ether, dioxane, tetrahydrofuran, and dichloromethane. Such transformations are well known in the chemical literature, and examples can be found in Greene, T. W., Wuts, P. G. M., “Protective Groups in Organic Synthesis”, John Wiley & Sons, Inc., 3^{rd} Edition, 1999.

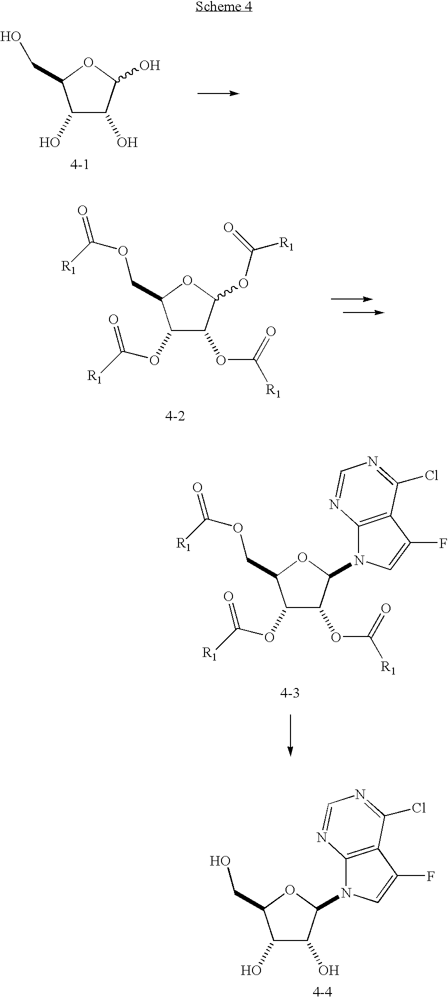

Intermediate 4-3 can be produced in a number of ways. In Scheme 4 the Vorbruggen reaction (“Synthesis of Nucleosides” by H. Vorbruggen and C. Ruh-Pohlenz in Organic Reactions, vol. 55, pp 1-630, 2000) is used to attach the nucleobase 5-fluoro-4-chloro-7H-pyrrolo[2,3-d]pyrimidine (1-4) to form the protected nucleoside 4-3. The ester protecting groups are then removed by any appropriate method, such as acid- or base-catalyzed hydrolysis and transesterification (for example, with sodium methoxide in methanol) to provide 4-4.

The C-2′ methyl group is next introduced as depicted in Scheme 5. In order to allow for orthogonal manipulation of the C-2′ hydroxyl group, the hydroxyl groups at positions 5′ and 3′ are first protected. This can be accomplished in a number of ways, and one of them is depicted in Scheme 5. The aforementioned reference, Greene, T. W., Wuts, P. G. M., “Protective Groups in Organic Synthesis”, John Wiley & Sons, Inc., 3^{rd} Edition, 1999, contains a number of examples; particularly suitable is the tetraisopropyldisiloxanylidene cyclic ether 5-1.

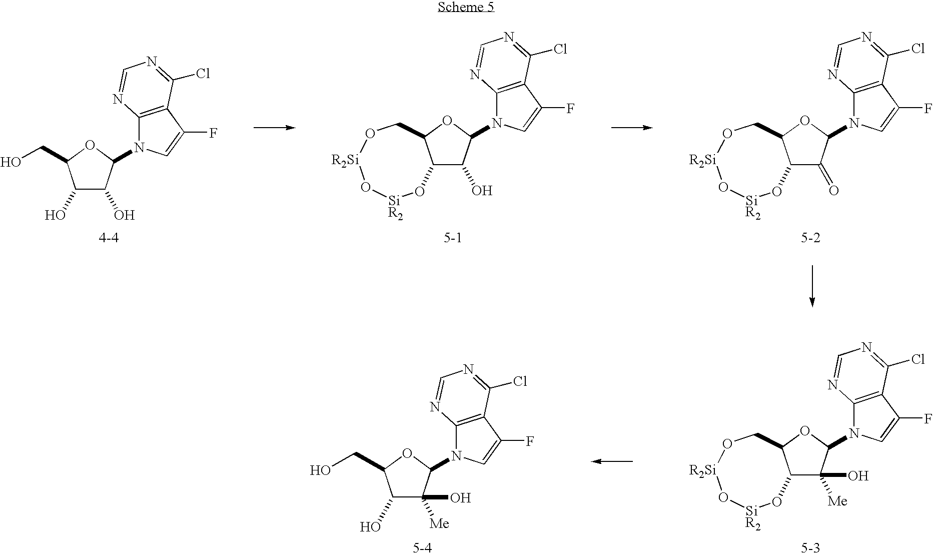

The remaining free hydroxyl group is then oxidized to a ketone 5-2 by using a suitable oxidation procedure, for example, the Swern or Moffatt oxidation or application of Dess-Martin periodinane. Examples of such processes can be found in the pertinent chemical literature, for example, in “Comprehensive Organic Transformations' by Richard C. Larock, published by VCH Publishers in 1989. The ketone 5-2 is then reacted with a suitable organometallic reagent, for example, methyl lithium and methylmagnesium halide. Such reactions are preferably performed at low temperatures and in an appropriate solvent, such as tetrahydrofuran and diethyl ether. In this instance, the methyl group is introduced from the less hindered face, and this steric control produces predominantly one isomer. Examples of such steric control can be found in the relevant chemical literature, for example, in “Stereochemistry of Organic Compounds”, by Ernest L. Eliel and Samuel H. Wilen, published by Wiley-Interscience Publications in 1994. The fluoro group is then introduced at the C-2′ position as depicted in Scheme 6.

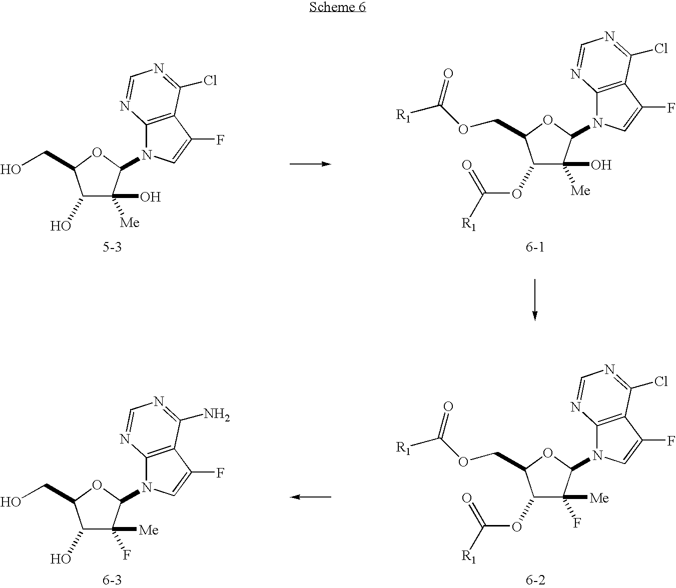

The hydroxyl groups present at positions 3′ and 5′ are selectively protected as described above. The use of lower alkyl or simple aromatic esters, such as acetate and benzoate, is advantageous. The selected reaction conditions are such that the sterically hindered C-2′ hydroxyl group remains unaffected. The introduction of fluorine is accomplished by the use of diethylaminosulfur trifluoride (DAST) or other suitable fluorination reagent optionally in the presence of a solvent, such as an aromatic hydrocarbon, tetrahydrofuran, and chloroform at low, ambient or elevated temperatures. An example of such transformation is described in US Patent Publication 2005/0009737 (published Jan. 13, 2005). The desired nucleoside 6-3 is then obtained by hydrolytic removal of the ester protecting groups, followed by displacement of the chlorine atom present in the nucleobase with ammonia. However, it is advantageous to perform the two last operations in one pot by use of ammonia in an appropriate solvent, such as methanol, at ambient or elevated temperature, using high pressure if needed.

### EXAMPLE 3

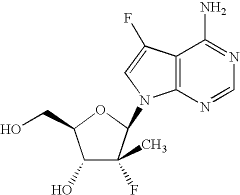

### 4-Amino-5-fluoro-7-(2-fluoro-2-C-methyl-β-D-ribofuranosyl)-7H-pyrrolo[2,3-d]pyrimidine

### Step A

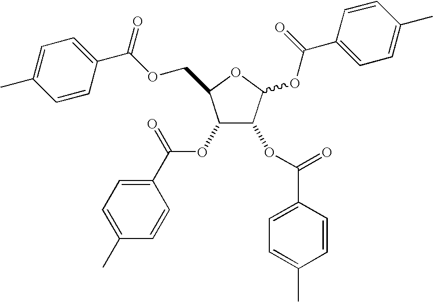

A solution of D-ribose (5.00 g, 33 mmol), triethylamine (46 mL, 330 mmol) and DMAP (810 mg, 6.6 mmol) in anhydrous DMF (80 mL) was treated dropwise with p-toluoyl chloride (22 mL, 165 mmol) and stirring was continued at ambient temperature for 3 h. The reaction was quenched by pouring onto 300 g of ice. After the ice had melted, the crude product was extracted into dichloromethane (3×100 mL), dried with anhydrous magnesium sulfate, filtered and the solvent was removed in vacuo. The remaining oily residue was triturated from acetone. The solvent was decanted, and the solid residue was crystallized from isopropyl alcohol (200 mL). The product was used in the next step.

### Step B

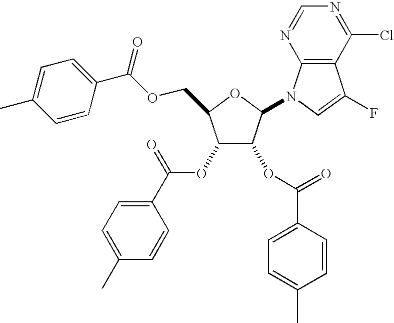

This compound is synthesized using a procedure such as that described by H. Vorbruggen and U. Niedballa in J. Ore. Chem., 39: 3654-3660 (1974). The 4-chloro-5-fluoro-7H-pyrrolo[2,3-d]pyrimidine (1-4) used in this transformation is prepared following the procedure described by A. B. Eldrup, et al. in J. Med. Chem., 47: 5284 (2004).

### Step C

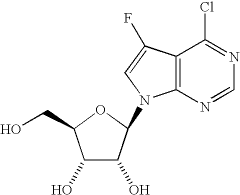

This compound is synthesized from the product of Step B following a procedure described by K. L. Smith, et al. in Bioorg. Med. Chem. Lett., 14: 3517-3520 (2004).

### Step D

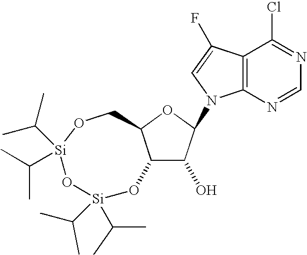

This compound is synthesized starting from the product of Step C using a procedure published by G. Gaubert, et al. in Tetrahedron Lett., 45: 5629-5632 (2004).

### Step E

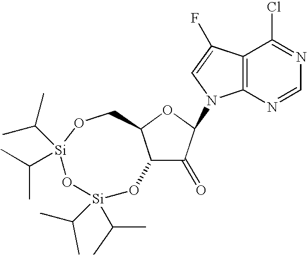

This compound is synthesized from the product of Step D following a procedure described by V. L. Moore, et al. in Biochemistry, 41: 14066-14075 (2002).

### Step F

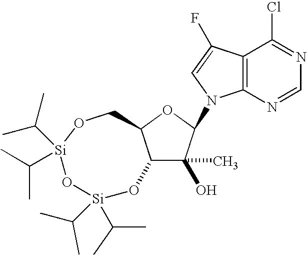

This compound is synthesized from the product of Step E following a procedure described by V. L. Moore, et al. in Biochemistry, 4: 14066-14075 (2002).

### Step G

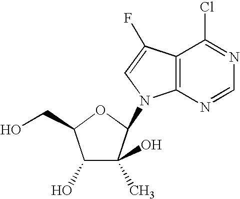

This compound is synthesized from the product of Step F using a procedure published by M. Gallo, et al. in Tetrahedron, 57: 5707-5713 (2001).

### Step H

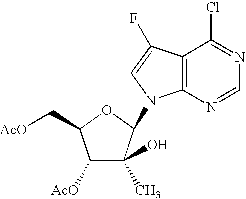

This diester is synthesized from the product of Step G using a procedure described by M. Akira, et al. in Chem. Pharm. Bull., 35: 3967-3970 (1987).

### Step I

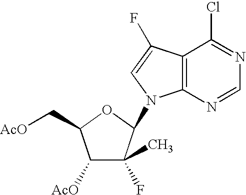

This compound is prepared by treating the product of Step H with DAST following the procedure described in US Patent Publication 2005/0009737.

### Step J

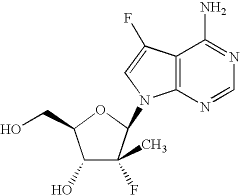

Example 3 is synthesized by treating the product of Step I with methanolic ammonia following conditions described for Example 62, Step F in U.S. Pat. No. 6,777,395, the contents of which are incorporated by reference in their entirety.

### EXAMPLE 4

### Nucleoside 5′-Triphosphates

The nucleoside 5′-triphosphates of the present invention were prepared according to the general procedures described in Chem. Rev. 100: 2047 (2000).

### EXAMPLE 5

### Purification and Purity Analysis of Nucleoside 5′-Triphosphates

Triphosphates were purified by anion exchange (AX) chromatography using a 30×100 mm Mono Q column (Pharmacia) with a buffer system of 50 mM Tris, pH 8. Elution gradients were typically from 40 mM NaCl to 0.8 M NaCl in two column volumes at 6.5 mL/min. Appropriate fractions from anion exchange chromatography were collected and desalted by reverse-phase (RP) chromatography using a Luna C18 250×21 mm column (Phenomenex) with a flow rate of 10 ml/min. Elution gradients were generally from 1% to 95% methanol in 14 min at a constant concentration of 5 mM triethylammonium acetate (TEAA).

Mass spectra of the purified triphosphates were determined using on-line HPLC mass spectrometry on a Hewlett-Packard (Palo Alto, Calif.) MSD 1100. A Phenomenex Luna (C18(2)), 150×2 mm, plus 30×2 mm guard column, 3-μm particle size was used for RP HPLC. A 0 to 50% linear gradient (15 min) of acetonitrile in 20 mM TEAA (triethylammonium acetate) pH 7 was performed in series with mass spectral detection in the negative ionization mode. Nitrogen gas and a pneumatic nebulizer were used to generate the electrospray. The mass range of 150-900 was sampled. Molecular masses were determined using the HP Chemstation analysis package.

The purity of the purified triphosphates was determined by analytical RP and AX HPLC. RP HPLC with a Phenomonex Luna or Jupiter column (250×4.6 mm), 5-μm particle size was typically run with a 2-70% acetonitrile gradient in 15 min in 100 mM TEAA, pH 7. AX HPLC was performed on a 1.6×5 mm Mono Q column (Pharmacia). Triphosphates were eluted with a gradient of 0 to 0.4 M NaCl at constant concentration of 50 mM Tris, pH 8. Purity of the triphosphates was generally >80%.

### Biological Assays

The assays employed to measure the inhibition of HCV NS5B polymerase and HCV replication are described below.

The effectiveness of the compounds of the present invention as inhibitors of HCV NS5B RNA-dependent RNA polymerase (RdRp) was measured in the following assay.

### A. Assay for Inhibition of HCV NS5B Polymerase:

This assay was used to measure the ability of the fluorinated pyrrolo[2,3-d]pyrimidine nucleoside triphosphates of the present invention to inhibit the enzymatic activity of the RNA-dependent RNA polymerase (NS5B) of the hepatitis C virus (HCV) on a heteromeric RNA template.

### Procedure:

### Assay Buffer Conditions: (50 μL-total/reaction)

20 mM Tris, pH 7.5
50 μM EDTA
5 mM DTT
2 mM MgCl₂
80 mM KCl
0.4 U/μL RNAsin (Promega, stock is 40 units/μL)
0.75 μg t500 (a 500-nt RNA made using T7 runoff transcription with a sequence from the NS2/3 region of the hepatitis C genome)
1.6 μg purified hepatitis C NS5B (form with 21 amino acids C-terminally truncated)
1 μM A, C, U, GTP (Nucleoside triphosphate mix)
[alpha-³²P]-GTP or [alpha-³³P]-GTP

The nucleoside triphosphates were tested at various concentrations up to 100 μM final concentration.

An appropriate volume of reaction buffer was made including enzyme and template t500. Nucleoside triphosphates of the present invention were pipetted into the wells of a 96-well plate. A mixture of nucleoside triphosphates (NTP's), including the radiolabeled GTP, was made and pipetted into the wells of a 96-well plate. The reaction was initiated by addition of the enzyme-template reaction solution and allowed to proceed at room temperature for 1-2 h.

The reaction was quenched by addition of 20 μL 0.5M EDTA, pH 8.0. Blank reactions in which the quench solution was added to the NTPs prior to the addition of the reaction buffer were included.

50 μL of the quenched reaction were spotted onto DE81 filter disks (Whatman) and allowed to dry for 30 min. The filters were washed with 0.3 M ammonium formate, pH 8 (150 mL/wash until the cpm in 1 mL wash is less than 100, usually 6 washes). The filters were counted in 5-mL scintillation fluid in a scintillation counter.

The percentage of inhibition was calculated according to the following equation: % Inhibition=[1−(cpm in test reaction−cpm in blank)/(cpm in control reaction−cpm in blank)]×100.

Representative compounds tested in the HCV NS5B polymerase assay exhibited IC₅₀'s less than 50 micromolar.

### B. Assay for Inhibition of HCV RNA Replication:

The compounds of the present invention were also evaluated for their ability to affect the replication of Hepatitis C Virus RNA in cultured hepatoma (HuH-7) cells containing a subgenomic HCV Replicon. The details of the assay are described below. This Replicon assay is a modification of that described in V. Lohmann, F. Korner, J-O. Koch, U. Herian, L. Theilmann, and R. Bartenschlager, “Replication of a Sub-genomic Hepatitis C Virus RNAs in a Hepatoma Cell Line,” Science 285:110 (1999).

### Protocol:

The assay was an in situ Ribonuclease protection, Scintillation Proximity based-plate assay (SPA). 10,000-40,000 cells were plated in 100-200 μL of media containing 0.8 mg/mL G418 in 96-well cytostar plates (Amersham). Compounds were added to cells at various concentrations up to 100 μM in 1% DMSO at time 0 to 18 h and then cultured for 24-96 h. Cells were fixed (20 min, 10% formalin), permeabilized (20 min, 0.25% Triton X-100/PBS) and hybridized (overnight, 50° C.) with a single-stranded ³³P RNA probe complementary to the (+) strand NS5B (or other genes) contained in the RNA viral genome. Cells were washed, treated with RNAse, washed, heated to 65° C. and counted in a Top-Count. Inhibition of replication was read as a decrease in counts per minute (cpm).

Human HuH-7 hepatoma cells, which were selected to contain a subgenomic replicon, carry a cytoplasmic RNA consisting of an HCV 5′ non-translated region (NTR), a neomycin selectable marker, an EMCV IRES (internal ribosome entry site), and HCV non-structural proteins NS3 through NS5B, followed by the 3′ NTR.

Representative compounds tested in the replication assay exhibited EC₅₀'s less than 50 micromolar.

### C. Assay for Intracellular Metabolism:

The compounds of the present invention were also evaluated for their ability to enter a human hepatoma cell line and be converted intracellularly into the corresponding nucleoside 5′-mono-, di-, and triphosphates.

Two cell lines, HuH-7 and HBI10A, were used for intracellular metabolism studies of the compounds of the present invention. HuH-7 is a human hepatoma cell line, and HBI10A denotes a clonal line derived from HuH-7 cells that harbors the HCV bicistronic replicon. HuH-7 cells were plated in complete Dulbecco's modified Eagle's medium containing 10% fetal bovine serum and HBI10A cells in the same containing G418 (0.8 mg/mL) at 1.5×10⁶ cells/60-mm dish such that cells were 80% confluent at the time of compound addition. Tritiated compound was incubated at 2 μM in the cell medium for 3 or 23 h. Cells were collected, washed with phosphate-buffered saline, and counted. The cells were then extracted in 70% methanol, 20 mM EDTA, 20 mM EGTA, and centrifuged. The lysate was dried, and radiolabeled nucleotides were analyzed using an ion-pair reverse phase (C-18) HPLC on a Waters Millenium system connected to an in-line β-RAM scintillation detector (IN/US Systems). The HPLC mobile phases consisted of (a) 10 mM potassium phosphate with 2 mM tetrabutylammonium hydroxide and (b) 50% methanol containing 10 mM potassium phosphate with 2 mM tetrabutyl-ammonium hydroxide. Peak identification was made by comparison of retention times to standards. Activity is expressed as picomoles of nucleotide detected in 106 HuH-7 or HBI10A cells.

The fluorinated pyrrolo[2,3-d]pyrimidine nucleoside compounds of the present invention were also evaluated for cellular toxicity and anti-viral specificity in the counterscreens described below.

### C. Counterscreens:

The ability of the fluorinated pyrrolo[2,3-d]pyrimidine nucleoside compounds of the present invention to inhibit human DNA polymerases was measured in the following assays.

a. Inhibition of Human DNA Polymerases Alpha and Beta:

### Reaction Conditions:

50 μL reaction volume

### Reaction Buffer Components:

### 20 mM Tris-HCl, pH 7.5

200 μg/mL bovine serum albumin

### 100 mM KCl

2 mM β-mercaptoethanol

### 10 mM MgCl₂

1.6 μM dA, dG, dC, dTTP

### α-³³P-dATP

### Enzyme and Template:

0.05 mg/mL gapped fish sperm DNA template
0.01 U/μL DNA polymerase α or β

### Preparation of Gapped Fish Sperm DNA Template:

Add 5 μL 1M MgCl₂ to 500 μL activated fish sperm DNA (USB 70076);
Warm to 37° C. and add 30 μL of 65 U/μL of exonuclease III (GibcoBRL 18013-011);

### Incubate 5 min at 37° C.;

Terminate reaction by heating to 65° C. for 10 min;
Load 50-100 μL aliquots onto Bio-spin 6 chromatography columns (Bio-Rad 732-6002) equilibrated with 20 mM Tris-HCl, pH 7.5;
Elute by centrifugation at 1,000×g for 4 min;
Pool eluate and measure absorbance at 260 nm to determine concentration.

The DNA template was diluted into an appropriate volume of 20 mM Tris-HCl, pH 7.5 and the enzyme was diluted into an appropriate volume of 20 mM Tris-HCl, containing 2 mM β-mercaptoethanol, and 100 mM KCl. Template and enzyme were pipetted into microcentrifuge tubes or a 96 well plate. Blank reactions excluding enzyme and control reactions excluding test compound were also prepared using enzyme dilution buffer and test compound solvent, respectively. The reaction was initiated with reaction buffer with components as listed above. The reaction was incubated for 1 hour at 37° C. The reaction was quenched by the addition of 20 μL 0.5M EDTA. 50 μL of the quenched reaction was spotted onto Whatman DE81 filter disks and air dried. The filter disks were repeatedly washed with 150 mL 0.3M ammonium formate, pH 8 until 1 mL of wash is <100 cpm. The disks were washed twice with 150 mL absolute ethanol and once with 150 mL anhydrous ether, dried and counted in 5 mL scintillation fluid.

The percentage of inhibition was calculated according to the following equation: % inhibition=[1−(cpm in test reaction−cpm in blank)/(cpm in control reaction−cpm in blank)]×100.

b. Inhibition of Human DNA Polymerase Gamma:

The potential for inhibition of human DNA polymerase gamma was measured in reactions that included 0.5 ng/μL enzyme; 10 μM dATP, dGTP, dCTP, and TTP; 2 μCi/reaction [α-³³P]-dATP, and 0.4 μg/μL activated fish sperm DNA (purchased from US Biochemical) in a buffer containing 20 mM Tris pH8, 2 mM β-mercaptoethanol, 50 mM KCl, 10 mM MgCl₂, and 0.1 μg/μL BSA. Reactions were allowed to proceed for 1 h at 37° C. and were quenched by addition of 0.5 M EDTA to a final concentration of 142 mM. Product formation was quantified by anion exchange filter binding and scintillation counting. Compounds were tested at up to 50 μM.

The percentage of inhibition was calculated according to the following equation: % inhibition=[1−(cpm in test reaction−cpm in blank)/(cpm in control reaction−cpm in blank)]×100.

The ability of the fluorinated pyrrolo[2,3-d]pyrimidine nucleoside compounds of the present invention to inhibit HIV infectivity and HIV spread was measured in the following assays.

c. HIV Infectivity Assay

Assays were performed with a variant of HeLa Magi cells expressing both CXCR4 and CCR5 selected for low background β-galactosidase (β-gal) expression. Cells were infected for 48 h, and β-gal production from the integrated HIV-1 LTR promoter was quantified with a chemiluminescent substrate (Galactolight Plus, Tropix, Bedford, Mass.). Inhibitors were titrated (in duplicate) in twofold serial dilutions starting at 100 μM; percent inhibition at each concentration was calculated in relation to the control infection.

d. Inhibition of HIV Spread

The ability of the compounds of the present invention to inhibit the spread of the human immunedeficiency virus (HIV) was measured by the method described in U.S. Pat. No. 5,413,999 (May 9, 1995), and J. P. Vacca, et al., Proc. Natl. Acad. Sci., 91: 4096-4100 (1994), which are incorporated by reference herein in their entirety.

The pyrrolo[2,3-d]pyrimidine nucleoside compounds of the present invention were also screened for cytotoxicity against cultured hepatoma (HuH-7) cells containing a subgenomic HCV Replicon in an MTS cell-based assay as described in the assay below. The HuH-7 cell line is described in H. Nakabayashi, et al., Cancer Res., 42: 3858 (1982).

e. Cytotoxicity Assay:

Cell cultures were prepared in appropriate media at concentrations of approximately 1.5×10⁵ cells/mL for suspension cultures in 3 day incubations and 5.0×10⁴ cells/mL for adherent cultures in 3 day incubations. 99 μL of cell culture was transferred to wells of a 96-well tissue culture treated plate, and 1 μL of 100-times final concentration of the test compound in DMSO was added. The plates were incubated at 37° C. and 5% CO₂ for a specified period of time. After the incubation period, 20 μL of CellTiter 96 Aqueous One Solution Cell Proliferation Assay reagent (MTS) (Promega) was added to each well and the plates were incubated at 37° C. and 5% CO₂ for an additional period of time up to 3 h. The plates were agitated to mix well and absorbance at 490 nm was read using a plate reader. A standard curve of suspension culture cells was prepared with known cell numbers just prior to the addition of MTS reagent. Metabolically active cells reduce MTS to formazan. Formazan absorbs at 490 nm. The absorbance at 490 nm in the presence of compound was compared to absorbance in cells without any compound added.

Reference: Cory, A. H. et al., “Use of an aqueous soluble tetrazolium/formazan assay for cell growth assays in culture,” Cancer Commun. 3: 207 (1991).

The following assays were employed to measure the activity of the compounds of the present invention against other RNA-dependent RNA viruses:

a. Determination of In Vitro Antiviral Activity of Compounds Against Rhinovirus (Cytopathic Effect Inhibition Assay):

Assay conditions are described in the article by Sidwell and Huffman, “Use of disposable microtissue culture plates for antiviral and interferon induction studies,” Appl. Microbiol. 22: 797-801 (1971).

### Viruses:

Rhinovirus type 2 (RV-2), strain HGP, was used with KB cells and media (0.1% NaHCO₃, no antibiotics) as stated in the Sidwell and Huffman reference. The virus, obtained from the ATCC, was from a throat swab of an adult male with a mild acute febrile upper respiratory illness.

Rhinovirus type 9 (RV-9), strain 211, and rhinovirus type 14 (RV-14), strain Tow, were also obtained from the American Type Culture Collection (ATCC) in Rockville, Md. RV-9 was from human throat washings and RV-14 was from a throat swab of a young adult with upper respiratory illness. Both of these viruses were used with HeLa Ohio-1 cells (Dr. Fred Hayden, Univ. of VA) which were human cervical epitheloid carcinoma cells. MEM (Eagle's minimum essential medium) with 5% Fetal Bovine serum (FBS) and 0.1% NaHCO₃ was used as the growth medium.

Antiviral test medium for all three virus types was MEM with 5% FBS, 0.1% NaHCO₃, 50 μg gentamicin/mL, and 10 mM MgCl₂.

2000 μg/mL was the highest concentration used to assay the compounds of the present invention. Virus was added to the assay plate approximately 5 min after the test compound. Proper controls were also run. Assay plates were incubated with humidified air and 5% CO₂ at 37° C. Cytotoxicity was monitored in the control cells microscopically for morphologic changes. Regression analysis of the virus CPE data and the toxicity control data gave the ED50 (50% effective dose) and CC50 (50% cytotoxic concentration). The selectivity index (SI) was calculated by the formula: SI=CC50÷ED50.

b. Determination of In Vitro Antiviral Activity of Compounds Against Dengue, Banzi, and Yellow Fever (CPE Inhibition Assay)

Assay details are provided in the Sidwell and Huffman reference above.

### Viruses:

Dengue virus type 2, New Guinea strain, was obtained from the Center for Disease Control. Two lines of African green monkey kidney cells were used to culture the virus (Vero) and to perform antiviral testing (MA-104). Both Yellow fever virus, 17D strain, prepared from infected mouse brain, and Banzi virus, H 336 strain, isolated from the serum of a febrile boy in South Africa, were obtained from ATCC. Vero cells were used with both of these viruses and for assay.

### Cells and Media:

MA-104 cells (BioWhittaker, Inc., Walkersville, Md.) and Vero cells (ATCC) were used in Medium 199 with 5% FBS and 0.1% NaHCO₃ and without antibiotics.

Assay medium for dengue, yellow fever, and Banzi viruses was MEM, 2% FBS, 0.18% NaHCO₃ and 50 μg gentamicin/mL.

Antiviral testing of the compounds of the present invention was performed according to the Sidwell and Huffman reference and similar to the above rhinovirus antiviral testing. Adequate cytopathic effect (CPE) readings were achieved after 5-6 days for each of these viruses.

c. Determination of In Vitro Antiviral Activity of Compounds Against West Nile Virus (CPE Inhibition Assay)

Assay details are provided in the Sidwell and Huffman reference cited above. West Nile virus, New York isolate derived from crow brain, was obtained from the Center for Disease Control. Vero cells were grown and used as described above. Test medium was MEM, 1% FBS, 0.1% NaHCO₃ and 50 μg gentamicin/mL.

Antiviral testing of the compounds of the present invention was performed following the methods of Sidwell and Huffman which are similar to those used to assay for rhinovirus activity. Adequate cytopathic effect (CPE) readings were achieved after 5-6 days.

d. Determination of In Vitro Antiviral Activity of Compounds Against Rhino, Yellow Fever, Dengue, Banzi, and West Nile Viruses (Neutral Red Uptake Assay)

After performing the CPE inhibition assays above, an additional cytopathic detection method was used which is described in “Microtiter Assay for Interferon: Microspectrophotometric Quantitation of Cytopathic Effect,” Appl. Environ. Microbiol. 31: 35-38 (1976). A Model EL309 microplate reader (Bio-Tek Instruments Inc.) was used to read the assay plate. ED50's and CD50's were calculated as above.

### Example of a Pharmaceutical Formulation

As a specific embodiment of an oral composition of a compound of the present invention, 50 mg of the compound of Example 1 or Example 2 is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for severity of the HCV infection. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of the structural formula I: 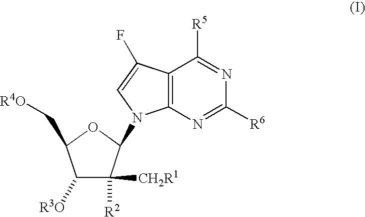
or a pharmaceutically acceptable salt thereof; wherein
R¹ is hydrogen or fluorine;
R² is fluorine or hydroxy;
R³ is hydrogen, C₁₋₁₆ alkylcarbonyl, C₂₋₁₈ alkenylcarbonyl, C₁₋₁₀ alkyloxycarbonyl, C₃₋₆ cycloalkylcarbonyl, C₃₋₆ cycloalkyloxycarbonyl, or an amino acyl residue of structural formula 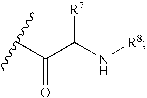
R⁴ is hydrogen, C₁₋₁₀ alkylcarbonyl, phosphoryl or a cyclic prodrug ester thereof, diphosphoryl, triphosphoryl, C₂₋₁₈ alkenylcarbonyl, C₁₋₁₀ alkyloxycarbonyl, C₃₋₆ cycloalkylcarbonyl, C₃₋₆ cycloalkyloxycarbonyl, CH₂O(C═O)C₁₋₄ alkyl, CH(C₁₋₄ alkyl)O(C═O)C₁₋₄ alkyl, an amino acyl residue of structural formula: 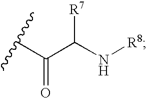
a residue of structural formula: 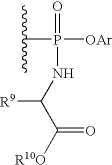
R⁵ is amino or hydroxy;
R⁶ is hydrogen, amino, or fluoro;
R⁷ is hydrogen, C₁₋₅ alkyl, or phenyl C₀₋₂ alkyl; and
R⁸ is hydrogen, C₁₋₄ alkyl, C₁₋₄ acyl, benzoyl, C₁₋₄ alkyloxycarbonyl, phenyl C₀₋₂ alkyloxycarbonyl, C₁₋₄ alkylaminocarbonyl, phenyl C₀₋₂ alkylaminocarbonyl, C₁₋₄ alkylsulfonyl, or phenyl C₀₋₂ alkylsulfonyl;
R⁹ is hydrogen, C₁₋₅ alkyl, phenyl or benzyl, wherein alkyl is unsubstituted or substituted with one substituent selected from the group consisting of hydroxy, methoxy, amino, carboxy, carbamoyl, guanidino, mercapto, methylthio, 1H-imidazolyl, and 1H-indol-3-yl and wherein phenyl and benzyl are unsubstituted or substituted with one to two substituents independently selected from the group consisting of halogen, hydroxy, and methoxy;
R¹⁰ is hydrogen, C₁₋₆ alkyl, C₃₋₆ cycloalkyl, phenyl, or benzyl, wherein alkyl and cycloalkyl are unsubstituted or substituted with one to three substituents independently selected from halogen, hydroxy, carboxy, C₁₋₄ alkoxy and wherein phenyl and benzyl are unsubstituted or substituted with one to three substituents independently selected from halogen, hydroxy, cyano, C₁₋₄ alkoxy, and trifluoromethyl; and
Ar is phenyl unsubstituted or substituted with one to three substituents independently selected from the group consisting of halogen, C₁₋₄ alkyl, C₁₋₄ alkoxy, C₁₋₄ alkylthio, cyano, nitro, amino, carboxy, trifluoromethyl, C₁₋₄ alkylamino, di(C₁₋₁₄ alkyl)amino, C₁₋₄ alkylcarbonyl, C₁₋₄ alkylcarbonyloxy, and C₁₋₄ alkyloxycarbonyl;
with the proviso that when R¹, R³, R⁴, and R⁶ are hydrogen and R² is hydroxy, then R⁵ cannot be amino.

2. The compound of claim 1 wherein R¹ is hydrogen; R² is hydroxy; and R³ and R⁴ are hydrogen.

3. The compound of claim 1 wherein R¹ is hydrogen; R² is fluoro; and R³ and R⁴ are hydrogen.

4. The compound of claim 1 which is 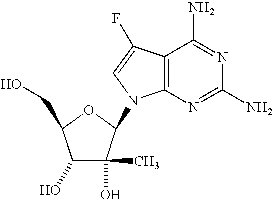
2,4-diamino-5-fluoro-7-(2-C-methyl-β-D-ribofuranosyl)-7H-pyrrolo[2,3-d]pyrimidine; 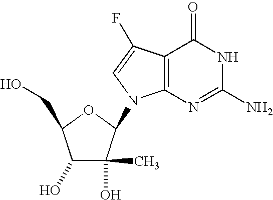
2-amino-5-fluoro-7-(2-C-methyl-β-D-ribofuranosyl)-7H-pyrrolo[2,3-d]pyrimidin-4(3H)-one; 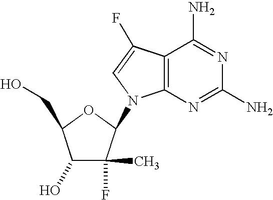
2,4-diamino-5-fluoro-7-(2-fluoro-2-C-methyl-β-D-ribofuranosyl)-7H-pyrrolo[2,3-d]pyrimidine; 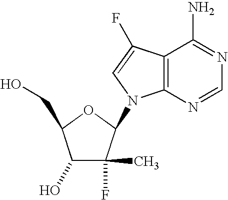
4-amino-5-fluoro-7-(2-fluoro-2-C-methyl-β-D-ribofuranosyl)-7H-pyrrolo[2,3-d]pyrimidine; or 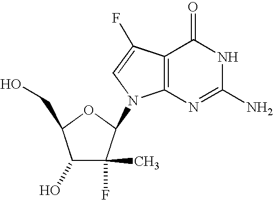
2-amino-5-fluoro-7-(2-fluoro-2-C-methyl-β-D-ribofuranosyl)-7H-pyrrolo[2,3-d]pyrimidin-4(3H)-one;
or a pharmaceutically acceptable salt thereof.

5. A pharmaceutical composition comprising a compound of claim 1 and a pharmaceutically acceptable carrier.

6-7. (canceled)

8. A method of treating hepatitis C virus (HCV) infection comprising administering to a mammal in need of such treatment a therapeutically effective amount of a compound according to claim 1.
